(19) United States
(12) Patent Application Publication
(10) Pub. No.: US 2026/0026913 A1
(43) Pub. Date: Jan. 29, 2026

(54) FALL PROTECTION FOR MEDICAL SUPPORT ARM

(71) Applicant: Mavig GmbH, Muenchen (DE)

(72) Inventors: Georg Muratidi, Oberaula (DE); Marc Hoffstetter, Muenchen (DE); Raphael Englert, Bad Hersfeld (DE); Lukas Zapf, Muenchen (DE); Alexander Buettner, Muenchen (DE)

(73) Assignee: Mavig GmbH, Muenchen (DE)

(21) Appl. No.: 19/206,656

(22) Filed: May 13, 2025

(30) Foreign Application Priority Data
Jul. 26, 2024 (EP) 24191154.4

(51) Int. Cl. A61B 90/50

(52) U.S. Cl. CPC A61B 90/50

(57) ABSTRACT

A support arm for receiving a medical device comprises at one of its ends a joint with a bore which is adapted to receive a pivot, so that the pivot can be rotated in the bore. The pivot is adapted to hold the medical device and is connected, in particular, to the medical device. The joint has a notch as a predetermined breaking point. The support arm also comprises a rope with a hook at one of its ends, which can be hooked into an opening in the joint. The notch is located between the opening and the bore. The other end of the rope can be connected to the pivot so that, in the connected state, the rope extends inside the bore. The rope comprises a means which is adapted to prevent the rope from twisting when the pivot is rotated in the bore.

The present disclosure relates to a catching device for securing a suspension means for medical devices and, in particular, to a fall protection for medical support arms.

In medical practices and clinics as well as medical-technical workstations in medical laboratories, objects of use are held in suspended positions of use in the area of workstations by means of support arms. Such support arms serving as suspension means are used, in particular, at medical workstations to keep ready medical objects of use such as X-ray shields, measuring devices, image reproduction devices (monitors), storage parts for working or treatment tools and the like.

Due to the sometimes heavy weight of the objects of use provided by means of the support arm and the frequent position and height adjustment of the mostly multi-part and long extension arms, signs of fatigue and subsequent breakage can occur at weak points. These include, in particular, articulated joints and suspension points. In order to prevent accidents and injuries to people staying in the area of the suspension means, it must therefore be ensured that the support arms are secured in the event of breakage in such a way that the breaking parts cannot fall to the ground.

DE 100 51 898 A1 describes a catching device for securing medical objects of use suspended from a suspension means against falling. The catching device comprises a safety loop which can be looped around a bolt of the suspension means for connection to the suspension means, wherein the safety loop has widenings at its ends, which engage through openings formed in a retaining body. Furthermore, the catching device comprises a main body which can be connected to a securing bolt of a connecting element, which is fixed to the object of use, by means of a securing pin. The main body can be detachably connected to the retaining body.

US 2022/0128193 A1 describes a catching device for a carrier system for preferably movably holding at least one medical device, with at least one carrier device having at least one critical point, and at least one elongate securing part. The carrier device comprises a first through opening in a first region, which is spaced apart from the critical point, and a second through opening in a second region, which is opposite the first region with respect to the critical point and is spaced apart from the critical point. The securing part is guided through both through openings, and the catching device comprises a first blocking element, which is attached to a first end of the securing part, and a second blocking element, which is attached to a second end of the securing part which is opposite the first end of the securing part. The two blocking elements are arranged in such a way that sliding of the securing part out of the two through openings is blocked.

It is an object of the present disclosure to provide an improved fall protection for a support arm for receiving a medical device, which can safely prevent the attached device from falling. Furthermore, the fall protection according to the disclosure should be structurally simple and, in particular, inexpensive to manufacture compared to the known solutions.

The present disclosure is defined in claim 1; the dependent claims describe embodiments of the disclosure.

The disclosure provides a support arm for receiving a medical device. The support arm comprises at one of its ends a joint with a bore which is adapted to receive a pivot so that the pivot is rotatable in the bore. The pivot is adapted to hold the medical device and is, in particular, connected to the medical device. The joint comprises a notch as a predetermined breaking point. The support arm also comprises a rope comprising a hook at one of its ends, which hook can be hooked into an opening of the joint. The rope is preferably a steel rope. The notch is located between the opening and the bore. The other end of the rope can be connected to the pivot so that, in the connected state, the rope extends inside the bore. The rope comprises a means that is adapted to prevent the rope from twisting when the pivot is rotated in the bore.

Due to the structure of the support arm according to the disclosure, the rope can ensure a mechanical connection between the support arm and the medical device.

The pivot can preferably be inserted axially into the bore. The hook is preferably a snap hook.

In particular, the pivot is adapted to hold an X-ray shield, a measuring device, an image reproduction device, a monitor or a storage part for working or treatment tools.

According to an embodiment, the means for preventing twisting can be configured in such a way that the rope is held by means of a stepped nipple in the pivot by an undercut.

Alternatively, the means for preventing twisting can have a swivel sleeve. In this case, the rope can be attached to the pivot by a loop using a bolt.

Compared to DE 100 51 898 A1 mentioned above, the construction of the support arm according to the disclosure is simpler, in particular because a complex milled part, which is required as a securing bolt in DE 100 51 898 A1, is unnecessary. Furthermore, according to DE 100 51 898 A1, the main body is connected to the securing bolt of a connection element, which is fixed to the article of use, by a pin. However, this pin cannot be monitored during assembly. In contrast, the hook of the present disclosure can be monitored when hooking into the opening of the joint, which increases the safety of the fall protection. In the case of the embodiment according to the disclosure with a swivel sleeve, space remains in the pivot, which can be used, for example, to pass cables therethrough.

In the case of US 2022/0128193 A1, the rope is attached to the opposite ends of the joint, with the predetermined breaking point in between. If, in the case of US 2022/0128193, the axial securing of the pivot in the joint fails or if this axial securing is inadvertently released or incorrectly fitted by the fitter, the attachment device will fall. In contrast, in the present disclosure, one end of the rope is arranged at the joint, while the other end is attached to the pivot of the attachment device. According to the disclosure, not only the predetermined breaking point is located therebetween, but also the fixing of the pivot in the joint. Thus, in the event of a separation of the pivot and the joint, a fall is intercepted in the disclosure, since the medical device is supported by the pivot via the rope and the hook on the joint.

In the following, the disclosure is explained in more detail with reference to the Figures, wherein

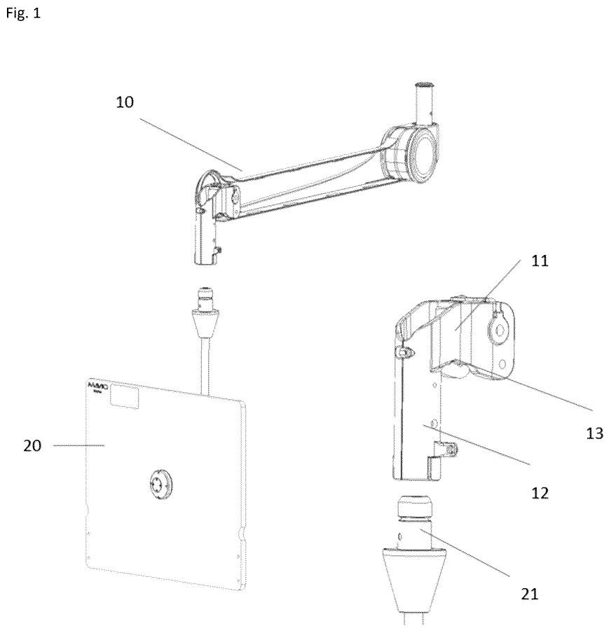

FIG. 1 schematically shows the structure of a support arm according to the present disclosure,

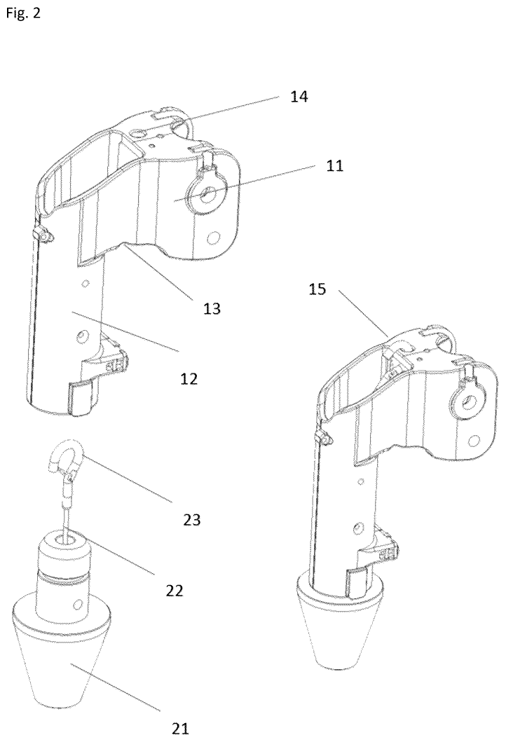

FIG. 2 shows the connection between support arm and pivot and

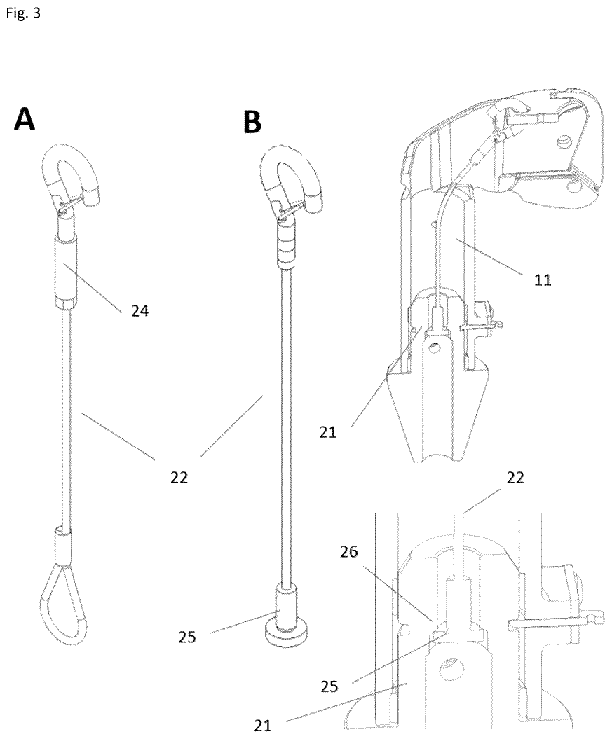

FIG. 3 shows the arrangement of the means for preventing twisting according to embodiments A and B.

FIG. 1 schematically shows the structure of the support arm according to the disclosure in accordance with an embodiment. In a known manner, the support arm 10 comprises a joint 11 which can be connected on one side to an extension arm, for example in order to pivotably attach the joint to the wall or ceiling of a treatment room, and which can be connected on the other side to a pivot 21 which is adapted to hold a medical device 20. To connect the pivot 21 to the joint 11, the joint 11 can have a bore 12 into which the pivot 21 can be inserted axially and held axially, for example, by a click mechanism in order to be rotatably connected to the joint 11.

At a defined point, the joint 11 comprises a notch 13 which represents a predetermined breaking point in the event of an overload due to the weight of the medical device 20 or due to excessive mechanical stress caused, for example, by a collision. In this case, the support arm 10 is secured to the medical device 20 against falling by means of a rope 22, in particular a steel rope, which will be described in more detail in the following.

As shown in FIG. 2, the pivot 21 is connected to a rope 22, at the end of which there is a hook 23, such as the snap hook shown. If the pivot 21 is inserted into the bore 12, the hook 23 can be hooked into an opening 14 of the joint 11, as shown with reference number 15 in FIG. 2. The opening 14 is arranged in the joint 11 in such a way that the notch 13, i.e. the predetermined breaking point, is located between the pivot 21 and the opening 14 for hooking in the hook 23, so that even if the joint 11 breaks at the predetermined breaking point, the part of the joint 11 connected to the support arm 10 remains connected to the pivot 21 holding the medical device and remains secured. In this way, a mechanical connection is created between the device 20 and the support arm 10, even if the predetermined breaking point breaks.

As a rule, it is desirable for the device 20 to be able to rotate around the pivot 21 without limit relative to the support arm 10. In order to prevent twisting and shearing of the rope 22, it must be equipped with a rotational degree of freedom. With reference to FIG. 3, two embodiments of such a means for preventing twisting are described.

According to embodiment A shown in FIG. 3, a loop 27 is arranged at the lower end of the rope 22, which is fastened in the pivot 21, for example by means of a bolt (not shown). The rope 22 is equipped with a rotating sleeve 24, which allows the pivot 21 to be rotated relative to the bore 12 of the joint 11 as desired.

In a structurally simpler and therefore more cost-efficient embodiment B, the rope 22 is provided with a stepped nipple 25 at the lower end. The stepped nipple 25 is held in the pivot 21 by an undercut 26. When the pivot 21 rotates relative to the joint 11, the rope 22 can rotate with the stepped nipple 25 relative to the pivot 21. This prevents the steel rope from twisting.

If the joint 11 is overloaded, the disclosure ensures that the device 20 attached to the pivot 21 is secured against falling, which can prevent or reduce the risk of accidents and damage to the medical device 20.

While various embodiments of the present disclosure have been described above, it should be understood that they are provided by way of example only, and not by way of limitation. The various features or configurations provided are intended to enable the person skilled in the art to understand exemplary features and functions of the present disclosure. The person skilled in the art will understand that the present disclosure is not limited to the shown features or configurations but may be implemented using a variety of alternative features and configurations. Furthermore, one or more features of one embodiment may be combined with one or more features of another of the described embodiments, as would be understood by a person skilled in the art. Therefore, the breadth and scope of the present disclosure should not be limited by any of the exemplary embodiments described above.

It is to be understood that in the present disclosure, the term “and/or” or the symbol “/” may include any and all combinations of one or more of the associated listed elements. For example, A and/or B and/or C includes any and all combinations of one or more of A, B, and C, including A, B, C, A and B, A and C, B and C, and a combination of A and B and C. Similarly, A/B/C includes any and all combinations of one or more of A, B and C, including A, B, C, A and B, A and C, B and C and a combination of A and B and C.

It is also understood that here any reference to an element with a designation such as “first”, “second” etc. does not generally restrict the set or order of these elements. Rather, these designations can be used here as a practical means of distinguishing between two or more elements or instances of an element. Thus, the reference to a first and a second element does not mean that only two elements can be used or that the first element must precede the second element in any way.

Various modifications of the implementations described in this disclosure are readily apparent to the person skilled in the art, and the general principles defined herein can be applied to other implementations without departing from the scope of the claims. Therefore, the disclosure is not limited to the implementations shown herein but is of the broadest possible scope consistent with the novel features and principles disclosed herein as recited in the claims below.

1. A support arm for receiving a medical device, wherein the support arm comprises at one of its ends a joint with a bore which is adapted to receive a pivot for the arrangement of the medical device, so that the pivot can be rotated in the bore, wherein the joint comprises a notch as a predetermined breaking point,
further comprising a rope which has a hook at one of its ends, which hook can be hooked into an opening of the joint, wherein the notch is arranged between the opening and the bore, wherein the other end of the rope can be connected to the pivot, so that in the connected state the rope) extends within the bore,
wherein the rope comprises a means which is adapted to prevent twisting of the rope when the pivot) rotates in the bore.

2. The support arm according to claim 1, wherein the rope is held by means of a stepped nipple in the pivot by an undercut.

3. The support arm according to claim 1, wherein the means for preventing twisting comprises a rotating sleeve.

4. The support arm according to claim 3, wherein the rope comprises a loop which is attached to the pivot by means of a bolt.

5. The support arm according to claim 1, wherein the pivot can be axially inserted into the bore.

6. The support arm according to claim 1, wherein the hook is a snap hook.

7. The support arm according to claim 1, wherein a mechanical connection between the support arm and the medical device is ensured by the rope.

8. The support arm according to claim 1, wherein the rope is a steel rope.

9. The support arm according to claim 1, wherein the pivot is adapted to hold an X-ray shield, a measuring device, an image reproduction device, a monitor, or a storage part for working or treatment tools.
